(19) United States
(12) Patent Application Publication
(10) Pub. No.: US 2022/0020035 A1
(43) Pub. Date: Jan. 20, 2022

(54) PERSONAL CARE PRODUCTS

(71) Applicant: Johnson & Johnson Consumer Inc., Skillman, NJ (US)

(72) Inventors: Honroe Swei, Monroe, NJ (US); Kurt Reynertson, Hopewell, NJ (US); Russell Gould, Yardley, PA (US); Russell Walters, Philadelphia, PA (US)

(21) Appl. No.: 17/414,860

(22) PCT Filed: Dec. 17, 2019
(86) PCT No.: PCT/IB2019/060955

§ 371 Date: Jun. 16, 2021

Related U.S. Application Data
(60) Provisional application No. 62/781,096, filed on Dec. 18, 2018.

(51) Int. Cl. G06Q 30/02; G06Q 30/06

(52) U.S. Cl. CPC G06Q 30/0201; G06Q 30/0621; G06Q 30/0631

(57) ABSTRACT

Methods of creating product stewardship profiles for ingredients in consumer products are disclosed. The methods may be employed by manufacturers of personal care products to design personal care products having desired product stewardship attributes. The methods may be employed by consumers when selecting personal care products that have consumer desired product stewardship attributes.

### FIELD OF THE INVENTION

The disclosure pertains to the manufacture of consumer goods.

### BACKGROUND OF THE INVENTION

Consumers, who are becoming more aware where the products they buy come from and of the social and environmental challenges being faced by the developing and developed world, are in search of ways to minimize their impact. The key areas of concern range from ethical hiring practices to respect for the environment. As more consumers become interested in the source of their products and the relationship between the products' manufacturers and their global partners, the demand that more products be “responsibly”, “ethically”, or “sustainably” sourced has increased.

Along the same lines, each individual consumer has its own values, needs and wants. The ability of a consumer to identify and systematically compare available alternatives while at the same time balancing its values, needs and wants would enable that consumer to make more informed decisions.

Given the overwhelming amount of conflicting information available on the internet, there is a growing need for a simple solution that can provide product and ingredient information that is not only user-friendly, but also reliable. An app that can be used at point-of-sale is a perfect solution, especially one that allows users to rapidly find information by, e.g., scanning the product's barcode with a smartphone.

Think Dirty® (www.thinkdirtyapp.com) is a mobile application and website that purports to educate consumers in order to make informed decisions regarding which cosmetic products to purchase. It uses a rating system called the “Dirty Meter” to rate ingredients and finished personal care products on a scale from 0-10, with 0 as the best indicating the product does not contain ingredients with a “documented potential negative health impact” and 10 as the worst indicating the product does contain ingredients with “potential serious negative long-term health effects.” Think Dirty® is also a store that connects app users directly to Amazon.com and other web outlets so that consumers can buy products featured in the app. It also provides a feature called “Our Picks,” which directs users to products similar to the one being searched that have a lower rating on the Dirty Meter. The website includes a link to the Think Dirty® Clean Boutique, where the consumer can make purchases from a small selection of Think Dirty® approved products Similar apps/websites include SkinDeep® by the Environmental Working Group (EWG) (http://www.ewg.org/skindeep), ToxFox by BUND (http://www.bund.net/chemie/toxfox), GoodGuide® by GoodGuide, Inc. (http://goodguide.com), SkinSAFE by HER Inc. (http://www.skinsafeproducts.com); Code Check by Code Check AG (http://play.google.com/store/apps/details?id=ch.ethz.im.codecheck&hl=en); GreenScreen® by Clean Production Action ((http://www.greenscreenchemicals.org/); and Ethical Barcode (http://play.google.com/store/apps/details?id=com.davidhampgonsalves.ethicalbarcode&hl=en).

While their intentions are good, websites and apps like those discussed above lack the rigor of peer-reviewed scientific evaluation. In addition, their ratings do not take into account important factors such as the purity of the ingredient, the amount of a specific ingredient in a formulation, and how the product is used. Each app uses unique methodology to rate products, and as a result a single product will likely have different ratings across apps. Consumers that do not have enough time to research the methodology of each app may have trouble distinguishing which apps, if any, are reliable. There currently is not an app that provides fully accurate ingredient information to consumers.

### SUMMARY OF THE INVENTION

Consumers want to make thoughtful decisions on what to do—and what not to do when purchasing personal care products. The invention is directed to a new, straightforward approach to understand stewardship tradeoffs at the product level. Current approaches are either straightforward but siloed (i.e., carbon footprint) or multidimensional but complex (i.e., lifecycle analysis). None have effectively incorporated benefit and impact tradeoffs. Also included in this invention is the idea that one size does not fit all. Consumers, retailers and/or brands each have their distinct stewardship profiles and needs.

By way of example, aspects of the present disclosure are directed to a method and framework to create ingredient profiles.

The present invention provides a number of benefits, including:
Providing a method and framework to create Product Stewardship Profiles (pSP) based on ingredients used.
Providing a method framework to create Consumer Stewardship Profiles (cSP) based on desired attributes of the consumer.
Providing a method framework to match cSP with target pSP to match desired traits with characteristics of product.
Providing a method and framework to design novel products (p′SP) specific to target pSP.
Providing a method and framework to design novel products (p′ SP) specific to target cSP.

### DESCRIPTION OF THE FIGURES

FIGS. 1(a)-1(m) show the methodology employed to define a product stewardship attribute profile for ingredients employed in personal care products in accordance with the invention.

FIG. 2 shows the methodology employed to define a product stewardship attribute profile (pSP) for a consumer product in accordance with the invention.

FIG. 3 shows the methodology employed to define a consumer stewardship attribute profile (cSP) for a consumer product in accordance with the invention.

FIG. 4 shows the methodology employed to combine multiple Stewardship Attributes (SA) into a composite Consumer Stewardship Attribute Rating (cSAR)n.

FIGS. 5(a) and 5(b) show the methodology employed to match cSP with target pSP to match desired traits with characteristics of personal care products.

FIGS. 6(a) and 6(b) show the methodology employed to design a novel product (p′ SP) against a target pSP profile.

FIGS. 7(a) and 7(b) show the methodology employed to design a novel product (p′ SP) against a target cSP profile.

FIG. 8 shows an example of the methodology of the invention employed on a personal care product.

FIG. 9 shows an example of the methodology of the invention employed on a personal care product in more detail.

FIG. 10 shows another example of the methodology of the invention employed on a personal care product.

### DETAILED DESCRIPTION OF THE INVENTION

### Definitions

iSARn(RAWi) means ingredient stewardship attribute rating of a particular raw material.

WT % n (RAWi) means the weight % in a product/formula of a particular raw material.

pSAn (Product Stewardship Attribute) means one discrete topical area relevant to product stewardship. Typically, a stewardship attribute is a set comprising multiple considerations on a related topical area.

pSARn (Product Stewardship Attribute Rating) means a discrete topical area relevant to product stewardship which is quantified.

pSP (Product Stewardship Profile) means the overall products stewardship attributes.

cSAn (Consumer Stewardship Attribute) means one discrete topical area relevant to consumer stewardship. Typically, a stewardship attribute is a set comprising multiple considerations on a related topical area.

cSARn (Consumer Stewardship Attribute Rating) means a discrete topical area relevant to consumer stewardship which is quantified.

cSARn(Indirectx) means Consumer Value Attribute rating obtained from objective behavior tracking.

cSARn(Directx) means Consumer Value Attribute rating obtained from questionnaires or surveys.

cSP (Consumer Stewardship Profile) means the overall consumers stewardship attributes.

Product (pn) means any relevant product.

Product (p′) means any novel product.

Product (i′) means any novel ingredient.

Target Product Profile (t) means target product and/or profile for matching or designing against.

pTSS (Product Total Stewardship Score) means the percentage match between pSP, cSP, and/or Target Product Profile.

Green chemistry is the design of chemical products and processes that reduce or eliminate the generation of hazardous substances. See, e.g., http://www.epa.gov/greenchemistry.

NGO means a non-governmental organization that is active in humanitarian, educational, healthcare, public policy, human rights, environmental and other areas to affect change according to their objectives.

A raw material is a basic material that is used to produce goods, finished products, energy, or intermediate materials which are themselves used to produce future finished products.

Stewardship means the careful and responsible management of something entrusted to one's care. Product stewardship means the responsible planning and management of resources during product development and commercialization.

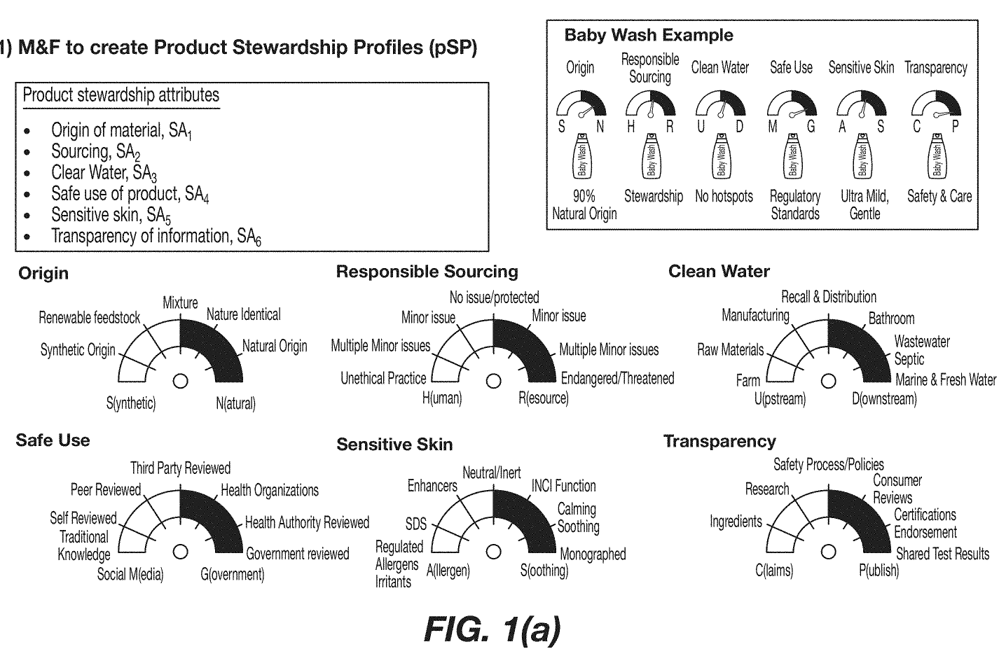

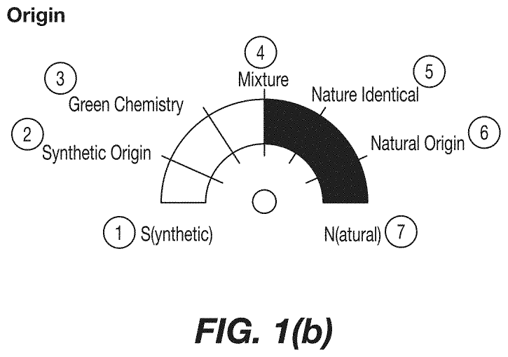

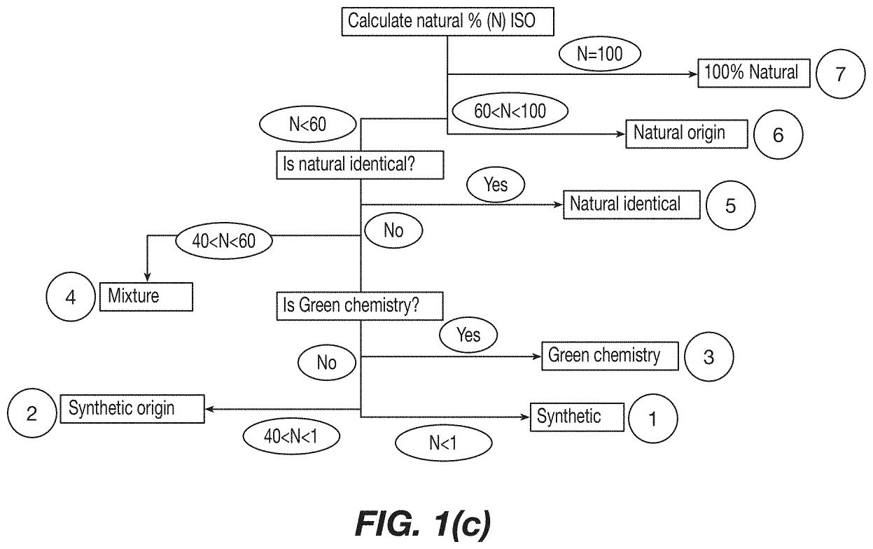

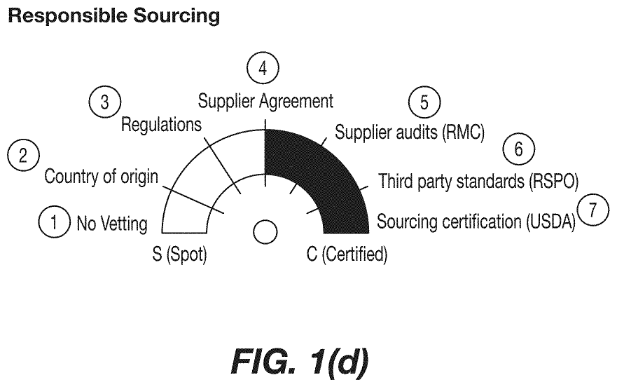

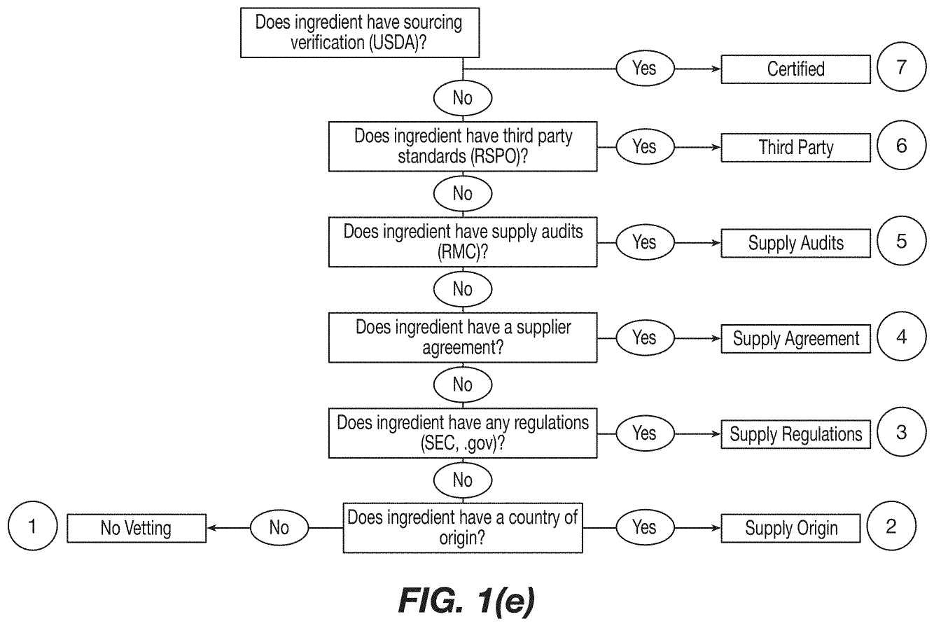

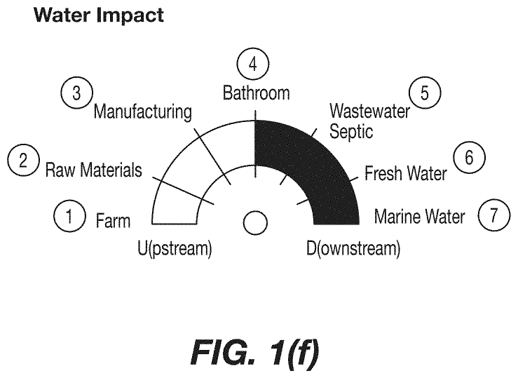

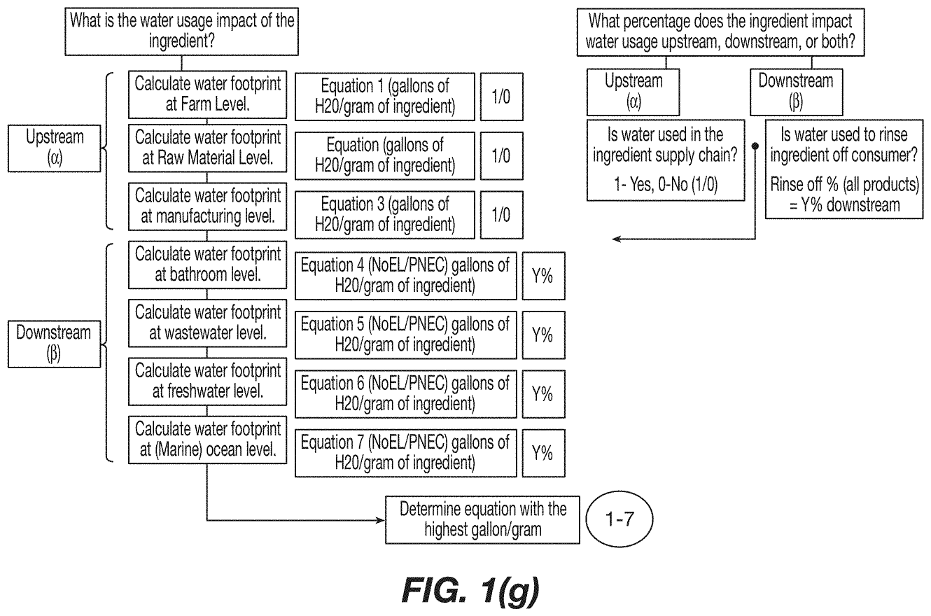

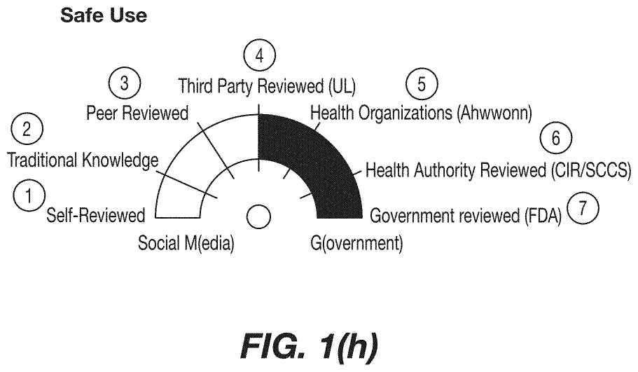

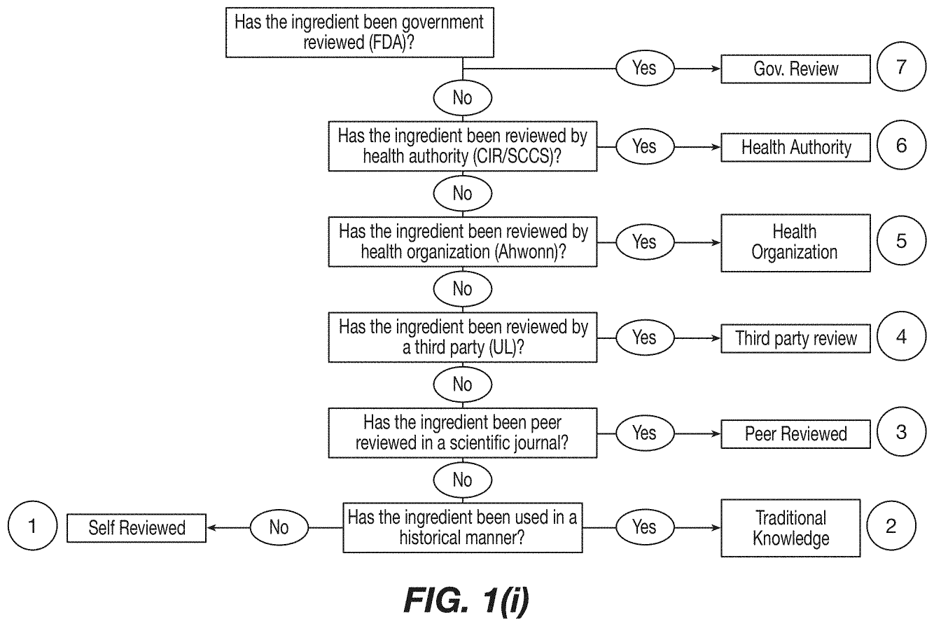

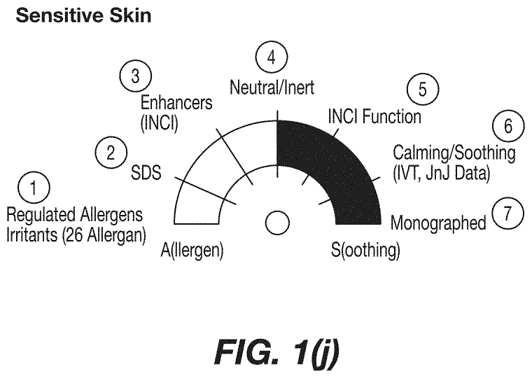

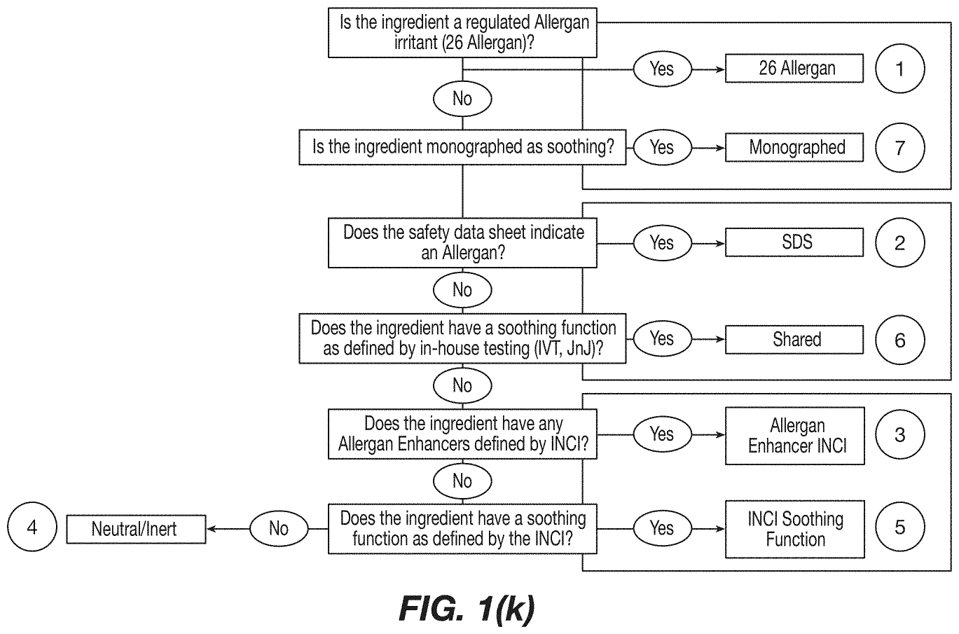

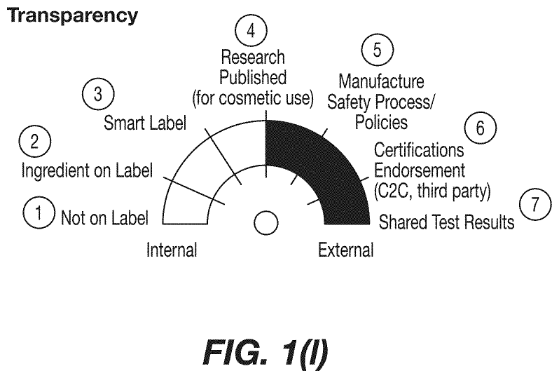

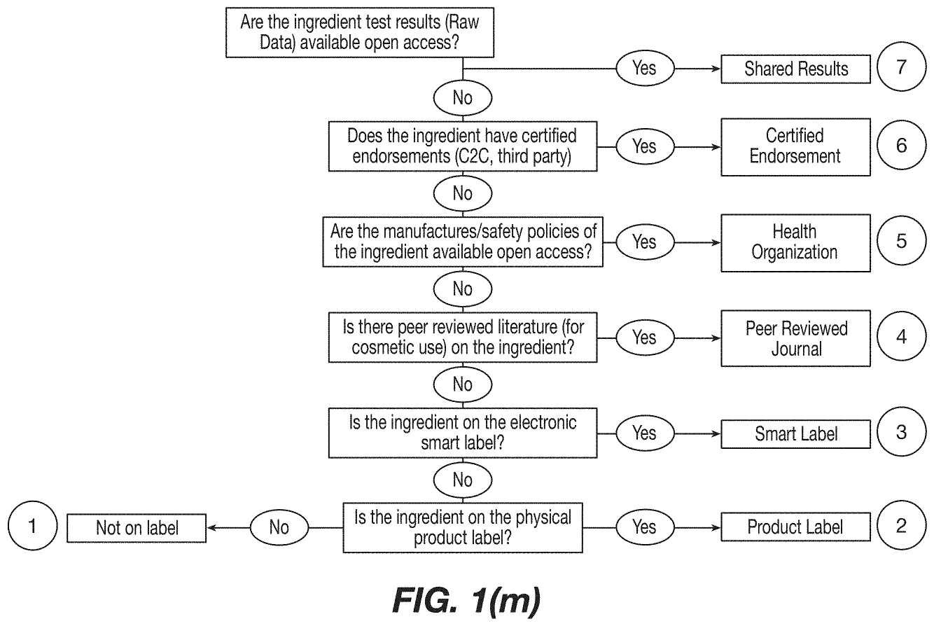

According to an embodiment, the invention is directed to a method and framework to define ingredient stewardship attribute ratings for raw materials commonly employed in personal care products. See FIG. 1(a). The methodology employed for each ingredient is shown in more detail in FIGS. 1(b)-1(m). SA are discrete topical areas relevant to consumer products, e.g.:
Origin, on a scale from S, synthetic to N, natural, wherein the ratings are as follows: (1) synthetic; (2) synthetic origin; (3) green chemistry: (4) mixture; (5) nature identical; (6) natural origin; and (7) natural.
Responsible sourcing, on a scale from S, spot (no vetting) to C, certified, wherein the ratings are as follows: (1) spot (no vetting); (2) country of origin; (3) regulations; (4) supplier agreement; (5) supplier audits; (6) third party standards; and (7) sourcing certification.
Clean water, on a scale from U, upstream to D, downstream, wherein the ratings are as follows: (1) farm; (2) raw materials; (3) manufacturing; (4) bathroom; (5) wastewater septic; (6) freshwater; and (7) marine.
Safe use, on a scale from M, social media to G, government, wherein the ratings are as follows: (1) self-reviewed; (2) traditional knowledge; (3) peer reviewed; (4) third party reviewed; (5) health organization reviewed; (6) health authority reviewed; and (7) government reviewed.
Sensitive skin, on a scale from A, allergen to S, soothing, wherein the ratings are as follows: (1) regulated allergens/irritants; (2) safety data sheet (SDS); (3) enhancers (INCI); (4) neutral/inert; (5) INCI function; (6) calming/soothing; and (7) monographed.
Transparency, on a scale from N, not publicly available to P, publicly available, wherein the ratings are as follows: (1) not on label; (2) on label; (3) digital label; (4) research published (for cosmetic use); (5) manufacture safety process and policies; (6) certifications/endorsement; and (7) shared test results.

The topical areas were selected based on the most common questions that the company received about its products; the assumption being that the questions reflected topics that were the most important to consumers.

Ingredients are placed on the spectrum of scenarios for each attribute and compared to a target scenario as determined via the methodology set forth in FIGS. 1(a)-1(m). Each of six Attributes are rated from (1)-(7). In essence, this aspect of the invention relates to a quantitative multi-dimensional means to define consumer's personal care product preferences.

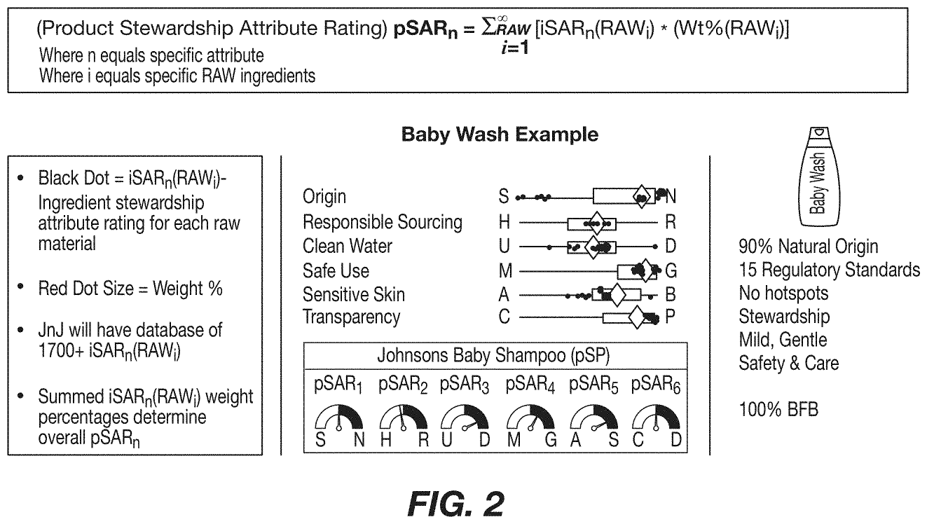

According to an embodiment, the invention is directed to a method and framework to define a product stewardship attribute profile (pSP) for any consumer product. See FIG. 2. This method involves the combination of multiple Stewardship Attributes (SA) into a composite Product Stewardship Attribute Rating (pSAR)n. See FIG. 2. The pSARn is calculated based on the weight summation of SA. See below.

$$( Product Stewardship Attribute Rating ) {pSAR}_{n} = {\sum }_{i = 1}^{∞} RAW [ {i SAR}_{n} ( RAWi ) * ( Wt \% ( RAWi ) ] \\ Where n equals specific attribute Where i equals specific RAW ingredients$$

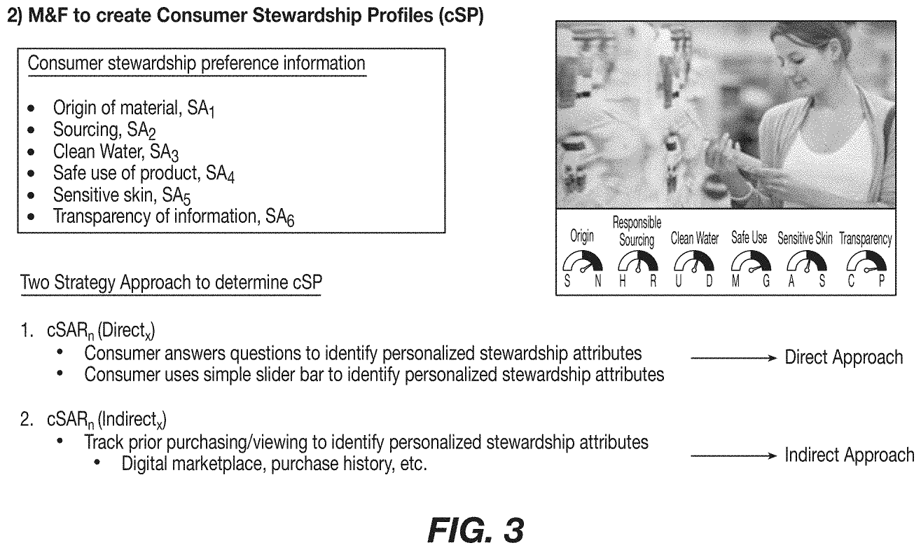

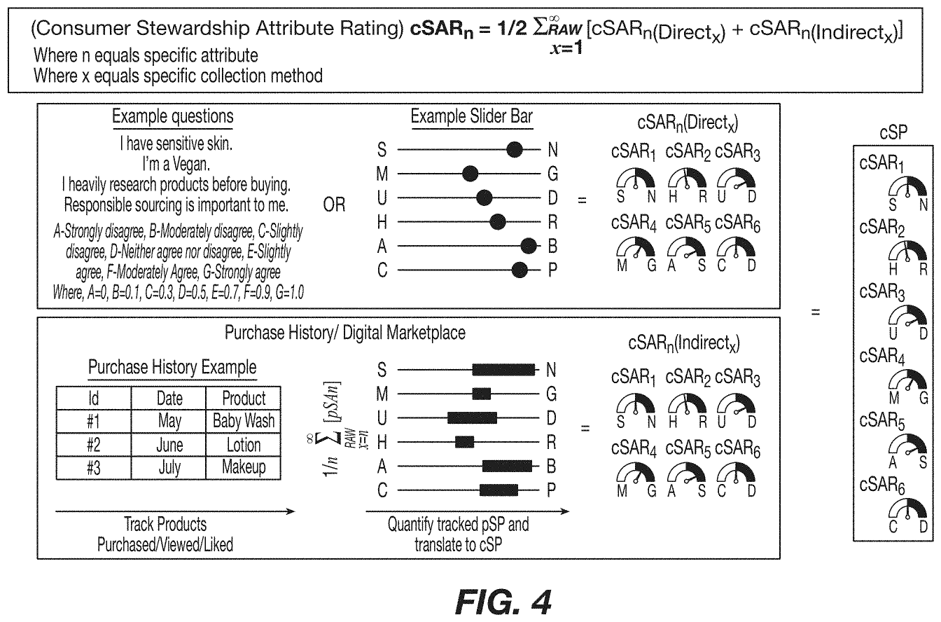

According to an embodiment, the invention is directed to a method and framework to define a consumer stewardship attribute profile (cSP). See FIG. 3. Using direct and indirect information from end users, a profile is constructed. Subjective information (e.g., questionnaires, direct user reports, etc.) and objective information (e.g., observed behavior) may be used separately or combined to build the complete profile. This method involves the combination of multiple Stewardship Attributes (SA) into a composite Consumer Stewardship Attribute Rating (cSAR)n. See FIG. 3. The cSARn is calculated based on the weight average of SA. See calculation below and FIG. 4. In essence, this aspect of the invention relates to a quantitative multi-dimensional means to define consumer stewardship preferences.

$$( Consumer Stewardship Attribute Rating ) {cSAR}_{n} = 1 \text{/} 2 {\sum }_{x = 1}^{∞} RAW [ {cSARn}_{( {Direct}_{x} )} + {cSARn}_{( {Indrect}_{x} )} ]$$

$$Where n equals specific attribute$$

$$Where x equals specific collection method$$

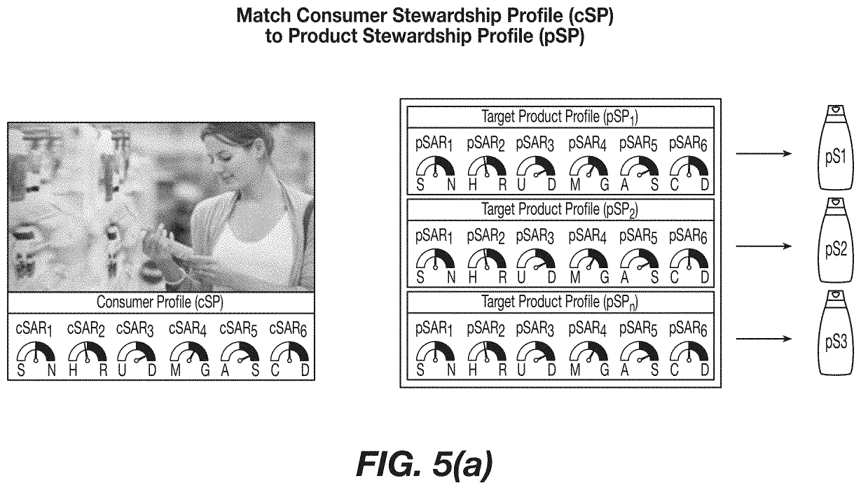

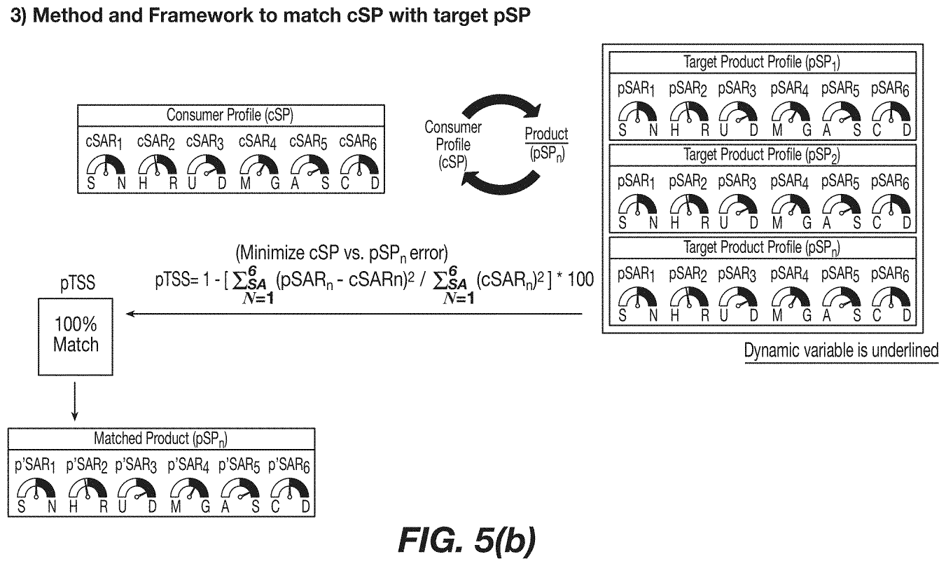

According to an embodiment, the invention is directed to a method and framework to match a cSP against a target pSP (as described in 1 and 2 above). See FIGS. 5(a) and 5(b). This method involves direct comparison of each cSAR and pSAR to determine error using a sum of squares approach. The percentage match of the cSP to the target pSP is then reported as the product total stewardship score (pTSS). Acceptance or rejection of the matched product is determined by stringent pTSS cutoffs. In essence, this aspect of the invention relates to a quantitative multi-dimensional means to optimize consumer choice.

According to an embodiment, a consumer can simply scan a barcode on a personal care product to determine whether the product meets its individual stewardship needs. The consumer can also receive suggestions of alternative products that meet these needs. According to this embodiment, a consumer downloads an app; logs in; builds its profile; uses a scanner to scan product; and finds out if a product is suitable. The consumer can also see if there are more suitable alternatives.

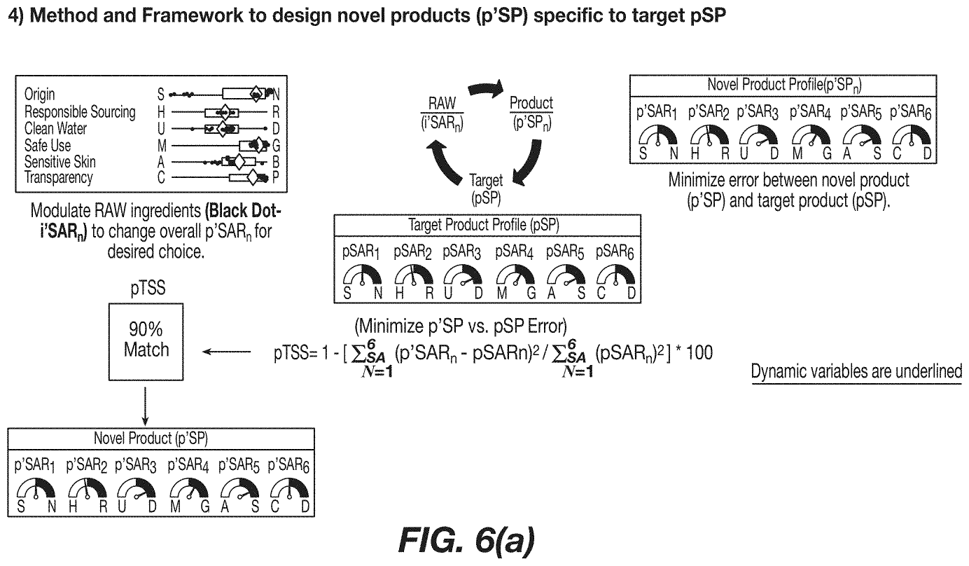

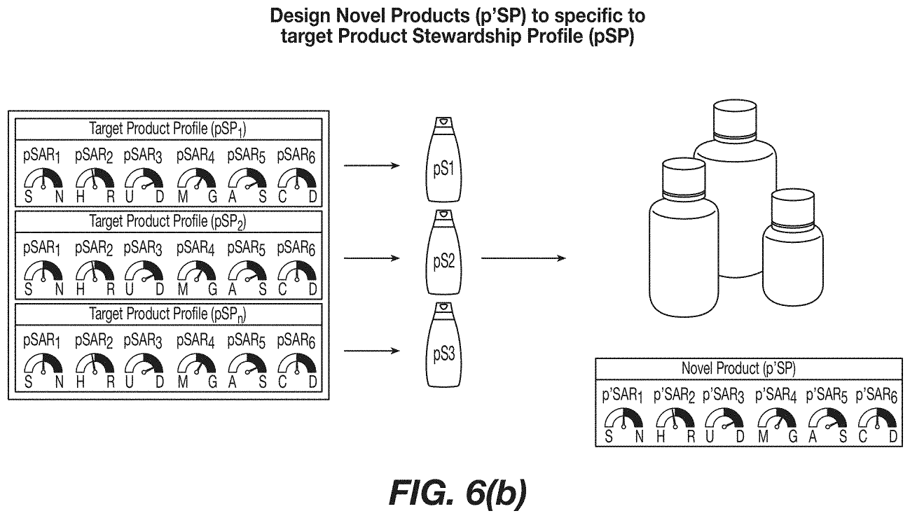

According to an embodiment, the invention is directed to a method and framework to design a novel product (p′ SP) against a target pSP profile (as described in 1 and 2 above). See FIGS. 6(a) and 6(b). This method involves direct comparison of each p′SAR and pSAR to determine error using a sum of squares approach. The percentage match of the p′ SP to the target pSP is then reported as the product total stewardship score (pTSS). Acceptance or rejection of the p′SP is determined by stringent pTSS cutoffs. In essence, this aspect of the invention relates to a quantitative multi-dimensional means to optimize product design.

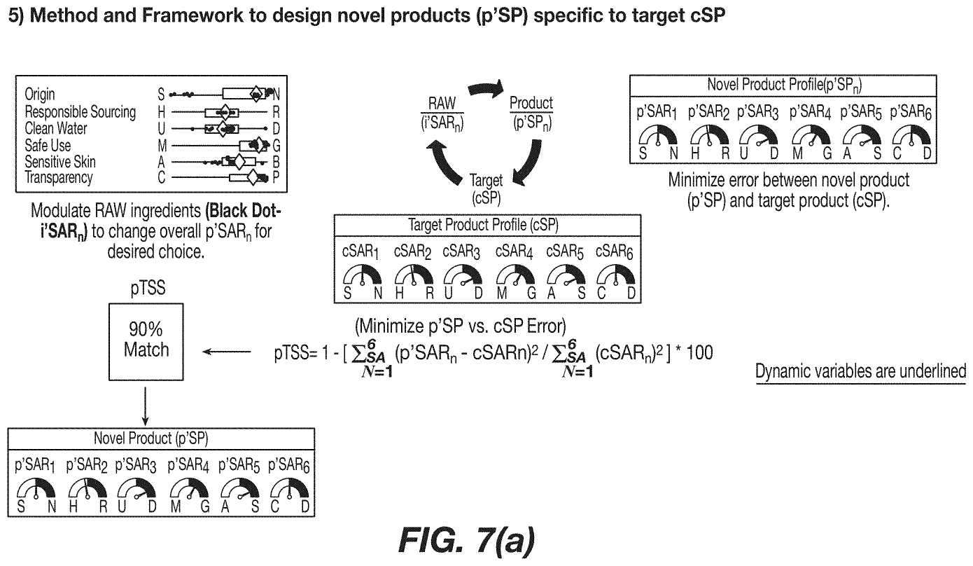

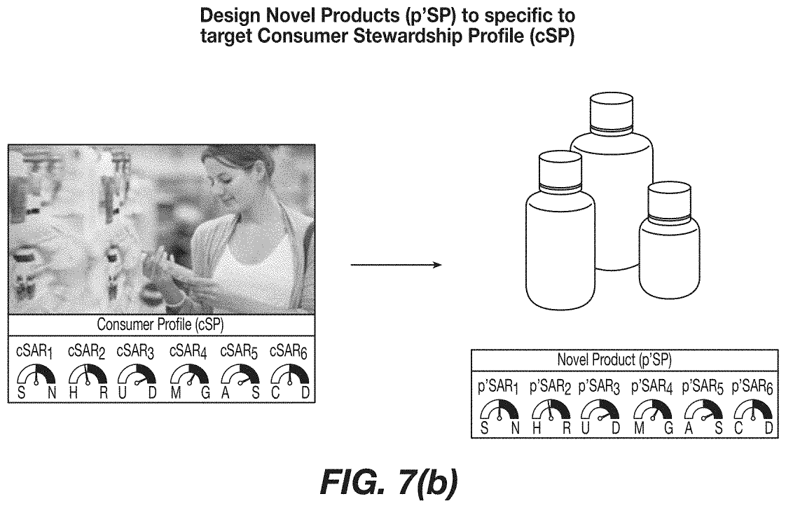

According to an embodiment, the invention is directed to a method and framework to design a novel product (p′ SP) against a target cSP (as described in 1 and 2 above). See FIG. 7. This method involves direct comparison of each p′SAR and cSAR to determine error using a sum of squares approach. The percentage match of the p′ SP to the target cSP is then reported as the product total stewardship score (pTSS). Acceptance or rejection of the p′SP is determined by stringent pTSS cutoffs. In essence, this aspect of the invention relates to a quantitative multi-dimensional means to optimize product design.

A number of private organizations have developed methodologies in an attempt to support natural claims, such as Natural Products Association, (NPA), Ecocert, Nature's International, and Nature. None of these have been accepted or endorsed by government agencies or competent authorities. While there are similarities, each of these uses a slightly different method for calculating natural or natural origin content. The net effect appears to be that while consumers continue to search for natural or natural origin, they are increasingly aware of criticisms around the use of natural claims and natural certifications.

ISO has adopted definitions per ISO/FDIS 16128:1, “Guidelines on technical definitions and criteria for natural & organic cosmetic ingredients and products”. These definitions were developed and agreed to by a wide range of stakeholders like NGO's, Academia and industry experts, including ASEAN Cosmetics Association, CASIC (Latin American Cosmetics, Toiletry and Perfumery Association), COSMOS-standard AISBL, Cosmetics Europe, IFOAM (International Foundation for Organic Agriculture), IFRA, and NATRUE.
A natural product is a chemical compound or substance produced by a living organism—that is found in nature. In the broadest sense, natural products include any substance produced by life.
Derived natural ingredients are cosmetics ingredients of greater than 50% by molecular weight natural origin, renewable carbon content, or any other relevant methods, obtained through defined chemical and/or biological processes with the intention of chemical modification.

Consumer products, natural or natural origin content should be substantiated following ISO/FDIS 16128:1 and ISO/WD 16128:2.
ISO/FDIS 16128-1 is a guide on technical definitions and criteria for Natural and Organic cosmetic ingredients and product.
ISO/WD 16128:2 No. 89 rev. builds on and enhances ISO/FDIS 16128-1, it describes approaches to determine indices that apply to the ingredient category.

In accordance with the invention, regulations, including ISO/FDIS 16128:1 and ISO/WD 16128:2 are followed in order to calculate the natural and organic content for cosmetic ingredients and/or products using the methodology set forth in FIGS. 1(a)-1(m).

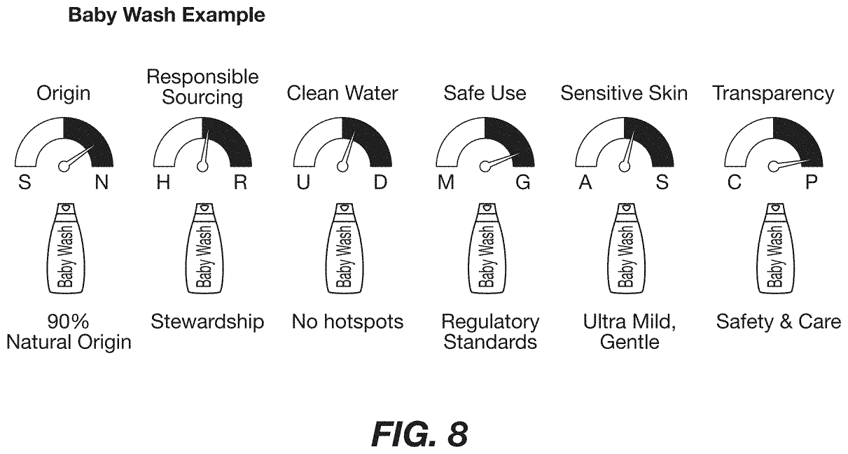

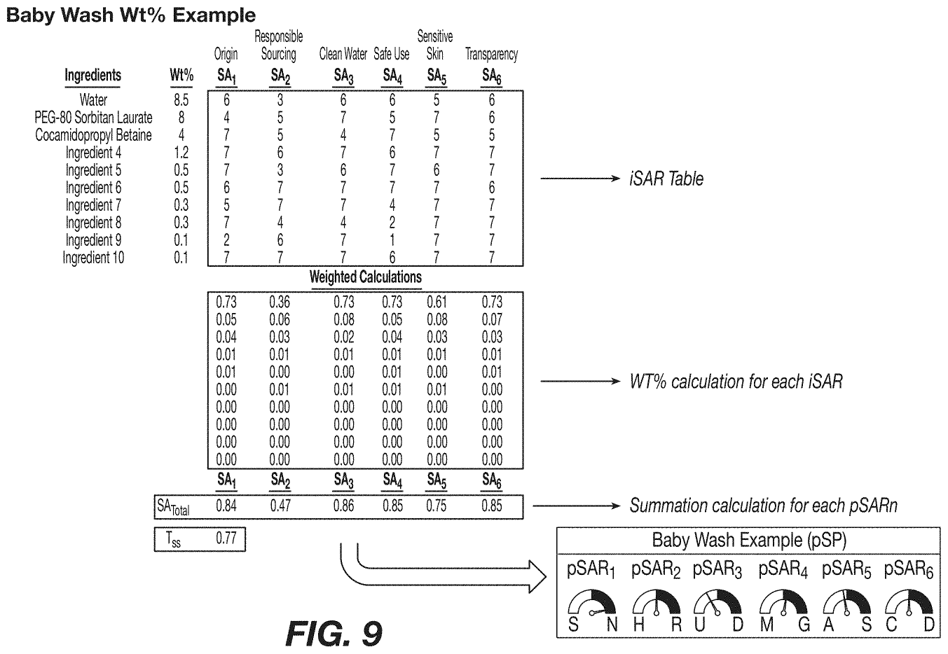

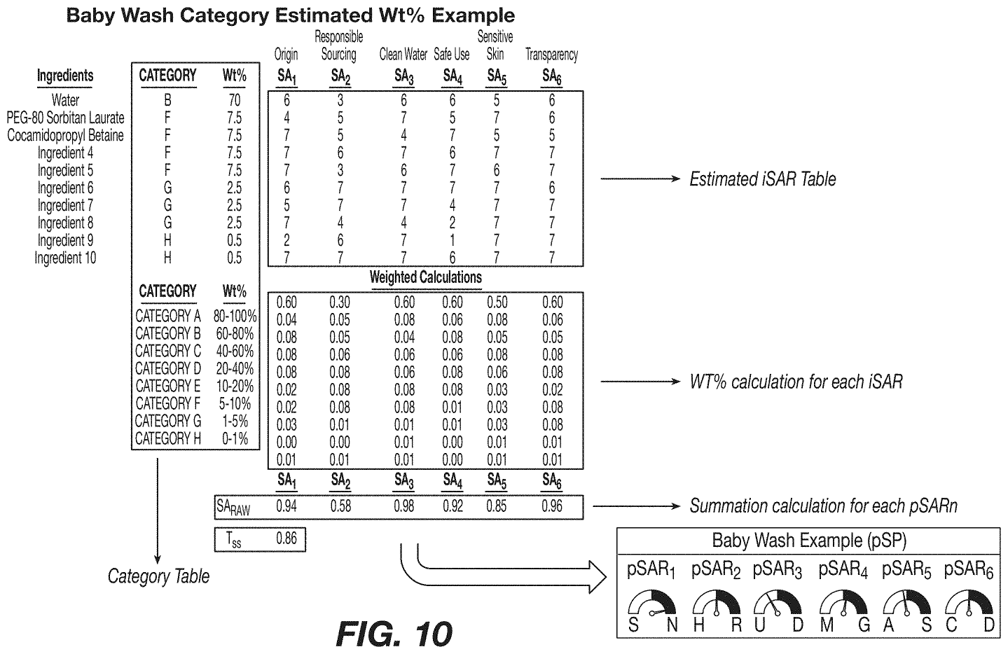

FIGS. 8-10 set forth an example wherein attributes of a Baby Wash are assessed in accordance with the invention. Numerical values are employed for each attribute for each ingredient and further assessed based on the weight percent of the ingredient in the formulation.

The invention serves as a unified way to serve the public in communicating the origin of ingredients used in products whether or not the ingredients are supported by certifications from third parties.

It will be understood that, while various aspects of the present disclosure have been illustrated and described by way of example, the invention claimed herein is not limited thereto, but may be otherwise variously embodied according to the scope of the claims presented in this and/or any derivative patent application.

1. (canceled)

2. (canceled)

3. (canceled)

4. (canceled)

5. (canceled)

6. (canceled)

7. A method to create an ingredient profile for an ingredient to be used in personal care products, comprising:
rating on a scale of 1-7 attributes for said ingredient, wherein said attributes are selected from the group consisting of origin of ingredient; sourcing of ingredient; impact said ingredient has on water; safety of said ingredient; impact of said ingredient on skin; and transparency with regard to publicly available information about said ingredient; and
combining the ratings to come up with a composite ingredient profile for said ingredient.

8. A method to create a product profile for a personal care product, comprising:
rating on a scale of 1-7 attributes for each ingredient in said product, wherein said attributes are selected from the group consisting of origin of ingredient; sourcing of ingredient; impact said ingredient has on water; safety of said ingredient; impact of said ingredient on skin; and transparency with regard to publicly available information about said ingredient;
combining the ratings for each ingredient to come up with a composite ingredient profile for each ingredient; and
combining the composite ingredient profile for all ingredients in said product to come up with a composite product profile for said product.

9. A method to create a consumer profile for preference of attributes of personal care products, comprising:
permitting said consumer to rate on a scale of 1-7 attributes for ingredients used in personal care products, wherein said attributes are selected from the group consisting of origin of ingredient; sourcing of ingredient; impact said ingredient has on water; safety of said ingredient; impact of said ingredient on skin; and transparency with regard to publicly available information about said ingredient; and
combining the ratings to come up with a composite consumer profile for preference.

10. A method to match a consumer profile with a product profile, comprising.
comparing consumer ratings for each attribute for ingredients used in personal care products with product profiles for each product in a set of products; and
determining a percentage match of the consumer profile to each product profile;
wherein the highest percentage match indicates a consumer preference for the product;
wherein said attributes are selected from the group consisting of origin of ingredient;
sourcing of ingredient; impact said ingredient has on water; safety of said ingredient; impact of said ingredient on skin; and transparency with regard to publicly available information about said ingredient.

11. A method to design a novel product having a desired product profile, comprising:
determining an attribute rating for each ingredient in a proposed novel product;
comparing the attribute rating for each ingredient in said proposed novel product to attribute ratings for ingredients in a target product profile;
determining an overall percentage match of the attribute ratings in the proposed novel product with the attribute ratings in a target product profile; and
modulating one or more ingredients in the proposed novel product to obtain a desired percentage match to the target product profile;
wherein said attributes are selected from the group consisting of origin of ingredient; sourcing of ingredient; impact said ingredient has on water; safety of said ingredient; impact of said ingredient on skin; and transparency with regard to publicly available information about said ingredient.

12. A method to design a novel product having a desired consumer profile, comprising:
determining an attribute rating for each ingredient in a proposed novel product;
comparing the attribute rating for each ingredient in said proposed novel product to attribute ratings for ingredients in a target consumer profile;
determining an overall percentage match of the attribute ratings in the proposed novel product with the attribute ratings in the target consumer profile; and
modulating one or more ingredients in the proposed novel product to obtain a desired percentage match to the target consumer profile if necessary;
wherein said attributes are selected from the group consisting of origin of ingredient; sourcing of ingredient; impact said ingredient has on water; safety of said ingredient; impact of said ingredient on skin; and transparency with regard to publicly available information about said ingredient.
